(19) United States
(12) Patent Application Publication
(10) Pub. No.: US 2020/0071714 A1
(43) Pub. Date: Mar. 5, 2020

(54) GENETICALLY ENGINEERING OF PLANT FIBRES AND PLANT THEREOF

(71) Applicant: LUXEMBOURG INSTITUTE OF SCIENCE AND TECHNOLOGY (LIST), Esch-sur-Alzette (LU)

(72) Inventor: Gea Guerriero, Esch/Alzette (LU)

(21) Appl. No.: 16/492,880

(22) PCT Filed: Mar. 6, 2018
(86) PCT No.: PCT/EP2018/055458

§ 371 Date: Sep. 10, 2019

(30) Foreign Application Priority Data
Mar. 10, 2017 (LU) LU100134

(51) Int. Cl. C12N 15/82

(52) U.S. Cl. CPC C12N 15/8226; C12N 15/8242

(57) ABSTRACT

The first object of the invention is directed to a genetic engineering process of plant comprising bast fibres, comprising the steps of (a) identification of the bast fibre promoter; and (b) amplification of the bast fibre promoter. The genetic engineering process of plant is remarkable in that it further comprises the step of preparing an expression cassette by fusing the bast fibre promoter with at least one gene coding for a first protein through a protein domain, the protein of the protein domain being a second protein different from the first protein.

### CROSS-REFERENCE TO RELATED APPLICATIONS

The present invention is the US national stage under 35 U.S.C. § 371 of International Application No. PCT/EP2018/055458, which was filed on Mar. 6, 2018, and which claims the priority of application LU 100134 filed on Mar. 10, 2017, the content of which (text, drawings and claims) are incorporated here by reference in its entirety.

### FIELD

The invention is directed to the genetic engineering of plant fibres.

### BACKGROUND

Hemp (Cannabis sativa L.) is grown for many uses, but is most widely cultivated for the bast (phloem) fibres in the stem. The stem has ‘two constituent parts: a cortex harbouring bundles of cellulosic fibres (or bast fibres) forming a sheath around a woody core called shiv, shive or hurd. The fibre is highly valued due to its length, strength and durability with a particular resistance to decay and has been heavily used for ropes, nets, sails and paper. Textile hemp is an economically important bast fibre-producing crop, with several applications in industry, namely the biocomposite, textile and construction sectors.

Bast fibres are defined as extraxylary sclerenchymatous elements obtained from the stem cortex of various plants and can be used as reinforcement for polymeric materials. Those fibres are composed primarily of cellulose which potentially has a Young's modulus of about 140 GPa, a value comparable with manmade aramid (Kevlar/Twaron) fibres (Summerscales J. et al., Composites: Part A, 2010, 41, 1329-1335).

The plants, other than the hemp, which are currently attracting most interests in the field of bast fibres are flax, jute (Corchorus), kenaf, sisal, ramie, cotton and nettle.

Arabinogalactan proteins (AGPs) are cell surface glycoproteins belonging to the hydroxyproline-rich glycoprotein superfamily which are involved in many aspects of plant development, among which cell wall-related processes in the bast fibres. One of the four main classes of these heavily glycosylated proteins is the fasciclin-like AGPs (FLAs). They are characterized by the occurrence of one or two AGP domains, as well as one or two fasciclin (FAS) domains and they constitute multigene families in plants.

A strong body of evidence in the scientific literature has highlighted the importance of FLAs in regulating aspects linked to cell wall biosynthesis, and, more generally, to stem mechanics in both herbaceous and woody species, as well as fibre growth.

### SUMMARY

The invention has for technical problem to provide plant fibres in which the properties can be tuned by genetic engineering. More in particular, the invention solves the problems of tuning the properties of a plant, or a plant fibre, without altering the intrinsic properties and the morphology of the plant fibres.

The first object of the invention is directed to a genetic engineering process of plant comprising bast fibres, comprising the steps of (a) identification of the bast fibre promoter; and (b) amplification of the bast fibre promoter. The plant genetic engineering process is remarkable in that it further comprises the step of preparing an expression cassette by fusing the bast fibre promoter with at least one gene coding for a first protein through a protein domain, the protein of the protein domain being a second protein different from the first protein.

According to a preferred embodiment, the protein domain is a cellulose-binding domain recognising xylan, preferentially the protein domain is SEQ ID NO:2 or SEQ ID NO:4.

According to a preferred embodiment, the bast fibre promoter is one promoter selected from the group of SEQ-ID NO:7, SEQ-ID NO:16, SEQ-ID NO:17, SEQ-ID NO:19, SEQ-ID NO:20, SEQ-ID NO:22, and SEQ-ID NO:23.

According to a preferred embodiment, the process further comprises the step of cloning the expression cassette in a first vector, preferentially the pENTR™/D-TOPO® vector.

According to a preferred embodiment, the process further comprises the following step of recombining the first vector, preferentially the pENTR™/D-TOPO® vector, into a second vector, preferentially the pEarleyGate 302 vector.

According to a preferred embodiment, the step (a) is performed by formation of complementary deoxyribonucleic acid libraries and subsequent high-throughput sequencing, the formation and sequencing being preferentially performed by using quantitative reverse transcription polymerase chain reaction (RT-qPCR).

According to a preferred embodiment, the step (a) is performed in the above snap-point part, ASP, and/or in the below snap-point part, BSP, of the stem of the fibrous plant.

According to a preferred embodiment, the step (b) is performed by means of polymerase chain reaction.

According to a preferred embodiment, the first protein is a surface-active protein, an elastomeric protein, a phosphoprotein or a spheroprotein.

According to a preferred embodiment, the surface-active protein is selected from the group of hydrophobins, chaplins, rodlins, and streptofactins, preferentially hydrophobins.

According to a preferred embodiment, the elastomeric protein is resilin.

According to a preferred embodiment, the phosphoprotein is casein.

According to a preferred embodiment, the spheroprotein is whey protein.

According to a preferred embodiment, the plant is selected from the group of flax, hemp, jute, kenaf, ramie and nettle, preferentially hemp.

According to a preferred embodiment, the process is performed by Agrobacterium tumefaciens GV3101.

The second object of the invention is directed to an expression cassette comprising a bast fibre promoter and at least one gene coding for a protein, characterized in that the bast fibre promoter is fused to a protein domain fused itself to the at least one gene coding for a first protein, the protein of the protein domain being a second protein different from the first protein.

According to a preferred embodiment, the protein domain is a cellulose-binding domain recognising xylan, preferentially the protein domain is SEQ ID NO:2 or SEQ ID NO:4.

The third object of the invention is directed to a plant comprising within its genetic code at least one expression cassette in accordance with the second object of the invention.

According to a preferred embodiment, the plant is selected from the group of flax, hemp, jute, kenaf, ramie and nettle, preferentially hemp.

The fourth object of the invention is directed to a transgenic seed of a plant, the seed comprising at least one expression cassette in accordance with the second object of the invention.

The invention is further directed to a fifth object, namely to a genetic engineering process of fibrous plant comprising bast fibres, comprising the steps of (a) identification of the bast fibre promoter; (b) amplification of the bast fibre promoter; and preparing an expression cassette by fusing the bast fibre promoter with at least one gene coding for a first protein, preferentially a gene coding for a surface-active protein. The process is remarkable in that the bast fibre promoter is one promoter selected from the group of SEQ-ID NO:7, SEQ-ID NO:16, SEQ-ID NO:17, SEQ-ID NO:19, SEQ-ID NO:20, SEQ-ID NO:22, and SEQ-ID NO:23.

According to a preferred embodiment, the process further comprises the step of cloning the expression cassette in a first vector, preferentially the pENTR™/D-TOPO® vector.

According to a preferred embodiment, the process further comprises the following step of recombining the first vector, preferentially the pENTR™/D-TOPO® vector, into a second vector, preferentially the pEarleyGate 302 vector.

According to a preferred embodiment, the step (a) is performed by formation of complementary deoxyribonucleic acid libraries and subsequent high-throughput sequencing, the formation and sequencing being preferentially performed by using quantitative reverse transcription polymerase chain reaction (RT-qPCR).

According to a preferred embodiment, the step (a) is performed in the above snap-point part, ASP, and/or in the below snap-point part, BSP, of the stem of the fibrous plant.

According to a preferred embodiment, the step (b) is performed by means of polymerase chain reaction.

According to a preferred embodiment, the first protein is a surface-active protein, an elastomeric protein, a phosphoprotein or a spheroprotein.

According to a preferred embodiment, the surface-active protein is selected from the group of hydrophobins, chaplins, rodlins, and streptofactins, preferentially hydrophobins.

According to a preferred embodiment, the elastomeric protein is resilin.

According to a preferred embodiment, the phosphoprotein is casein.

According to a preferred embodiment, the spheroprotein is whey protein.

According to a preferred embodiment, the plant is selected from the group of flax, hemp, jute, kenaf, ramie and nettle, preferentially hemp.

According to a preferred embodiment, the process is performed by Agrobacterium tumefaciens GV3101.

The invention is further directed to a sixth object, namely to an expression cassette comprising a bast fibre promoter and at least one gene coding for a protein, characterized in that the bast fibre promoter is one promoter selected from the group of SEQ-ID NO:7, SEQ-ID NO:16, SEQ-ID NO:17, SEQ-ID NO:19, SEQ-ID NO:20, SEQ-ID NO:22, and SEQ-ID NO:23.

The seventh object of the invention is directed to a plant comprising within its genetic code at least one expression cassette in accordance with the sixth object of the invention.

According to a preferred embodiment, the plant is selected from the group of flax, hemp, jute, kenaf, ramie and nettle, preferentially hemp.

The eighth object of the invention is directed to a transgenic seed of a plant, the seed comprising at least one expression cassette in accordance with the sixth object of the invention.

In general, the particular embodiments of each object of the invention are also applicable to other objects of the invention. To the extent possible, each object of the invention is combinable with other objects.

The invention is particularly interesting in that the genetically modified plants produce a recombinant protein. Thus the intrinsic properties of the plants are modified. As no chemical treatments are involved, no alteration in the fibre morphology of the plant is detected. Manufacture of new materials with enhanced and tunable properties is thus expected from this invention.

### DRAWINGS

FIG. 1 is an exemplary illustration of a sampling strategy of the stem tissues.

FIG. 2A is an exemplary illustration of an hierarchical clustering of expression profiles of 22 CsaFLAs in the three regions of stem.

FIG. 2B is an exemplary illustration of a rescaled expression values (calculated by subtracting to each gene expression value the average among the three regions and dividing by the standard deviation). Data in the form of bar graph are shown in FIG. 3.

FIGS. 3A and 3B is an exemplary illustration of an expression analysis of 22 CsaFLAs in fibres (A) and stem core (B) of C. sativa. Error bars indicate the standard error of the mean (n=4). Different letters indicate statistically significant values at the one-way ANOVA test (p<0.05).

FIG. 4 is an exemplary illustration of a PCR process between the bast-fibre promoter, the gene for cellulose-binding domain recognizing xylan and the gene of interest.

FIG. 5 is an exemplary illustration of a PCR process between the bast-fibre promotor and the gene of interest.

FIG. 6 is an exemplary illustration of a screening of transgenic tobacco plants transformed with the cassette CBM35-RodA under the control of the 35S promoter (in the vector pEarleyGate103).

### DETAILED DESCRIPTION

In order to provide plant fibres which present interesting properties without altering the intrinsic mechanical properties and morphology of the plant fibres, the technique of genetic engineering is employed. The goal is to provide a fibre which can produce interesting proteins, to confer interesting properties such as fire resistance properties, coloration change properties, hydrophobicity properties, and/or a combination thereof. In other words, transgenic fibre crops, capable of secreting surface-active proteins, in particular an elastomeric protein, a phosphoprotein or a spheroprotein, more particularly hydrophobins but also chaplins, rodlins, and streptofactins, are created. A gene of interest must therefore be present in the expression cassette.

The fibre crops are selected from the group consisting of flax, hemp, jute, kenaf, sisal, ramie, cotton and nettle. Hemp is used as the plant of choice, in the light of its wide industrial applications.

To perform the genetic engineering of the fibre crops and, more specifically to achieve the expression of the active proteins in the bast fibres, the promoter of the gene expressed in the bast fibres of the fibre crops is identified. It is thus necessary to identify marker genes for bast fibre thickening, in order to use their promoters to drive expression of the transgene. It is desirable to express the foreign gene during fibre thickening, to avoid possible interference during the elongation phase of the fibre cells. The preferential expression of the genes during this stage will guarantee that the fibres can carry out water/solute exchanges necessary for turgor pressure maintenance during active elongation.

In other words, it is desirable to identify one bast fiber promotor driving the expression of the transgene during the thickening phase of bast fibres (or that is active during a specific stage (for instance, the thickening phase) of the bast fibre development) and to amplify the bast fiber promotor.

Quantitative reverse transcription polymerase chain reaction (RT-qPCR) is then carried out on fibres separated from top and bottom internodes (plants aged of 1 month) on the fibre crops, in particular hemp.

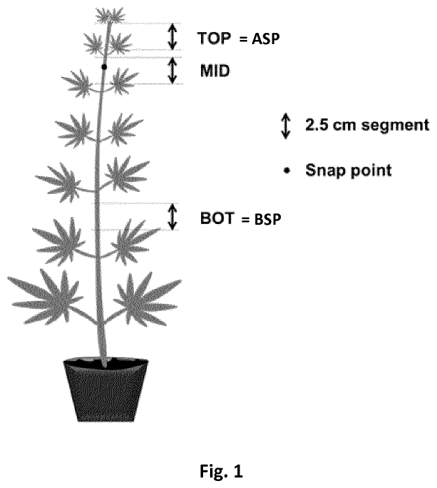

A stem of fibre crops can be divided into different zones, separated by the snap-point (i.e., an empirically-defined reference region marking the transition from elongation to secondary cell wall thickening). A first zone is the ASP part, namely the Above Snap-Point part, also referred as the TOP part on FIG. 1. It is the region right below the apex of the plant. A second zone is a zone comprising the snap point and is referenced as the MID (“middle”) part on FIG. 1. A third zone is the BSP, namely the Below Snap-Point part, also referred as the BOT (“bottom”) part on FIG. 1.

The separation between the top and bottom will thus enable the identification of genes enriched in two different stages of fibre formation, i.e. elongation and thickening, respectively.

A segment of 2.5 cm is collected in the middle of each internode to avoid too much variation in gene expression, due to the varying developmental stages of the cell types.

RNA is extracted from the collected bast fibres (three biological replicates, each consisting of a pool of 8-10 plants, showing homogeneous height, stem thickness and number of internodes).

The promoter of the gene of the bast fibers is best marked in the BSP part, because the bottom of the hemp undergoes girth increase (i.e. secondary growth), while the top of the hemp elongates rapidly.

It is preferable to select genes expressed in the bast fibres coming from the BSP part of the hemp because one does not want to interfere with the elongation of the fibres.

Identification of Bast Fibres Promoters Using Bioinformatics

In order to identify the FLA genes (which code for AGPs) in C. sativa (hereafter referred to as CsaFLAs (in italics for the genes), different databases were searched: the Medicinal Plant Genomics Resource (MPGR) (http://medicinalplantgenomics.msu.edu/mpgr_external_blast.shtml) and the Cannabis sativa Genome Browser Gateway (http://genome.ccbr.utoronto.ca/cgi-bin/hgGateway). CsaFLAs were identified by using orthologous FLA protein sequences of Arabidopsis thaliana and Populus trichocarpa. These sequences were used to perform a BLAT analysis (pairwise sequence alignment algorithm) against the hemp Finola and Purple Kush database (Cannabis Genome Browser Gateway) and a BLASTP in the MPGR database. Several incomplete sequences were retrieved when using the MPGR database; however it was possible to deduce their full length sequences either by querying the Cannabis Genome Browser Gateway, or the EST database at NCBI (dbEST; available at http://www.ncbi.nlm.nih.gov/dbEST/). The retrieved nucleotide sequences with the corresponding proteins are indicated in SEQ-ID NO:5 to SEQ-ID NO:27.

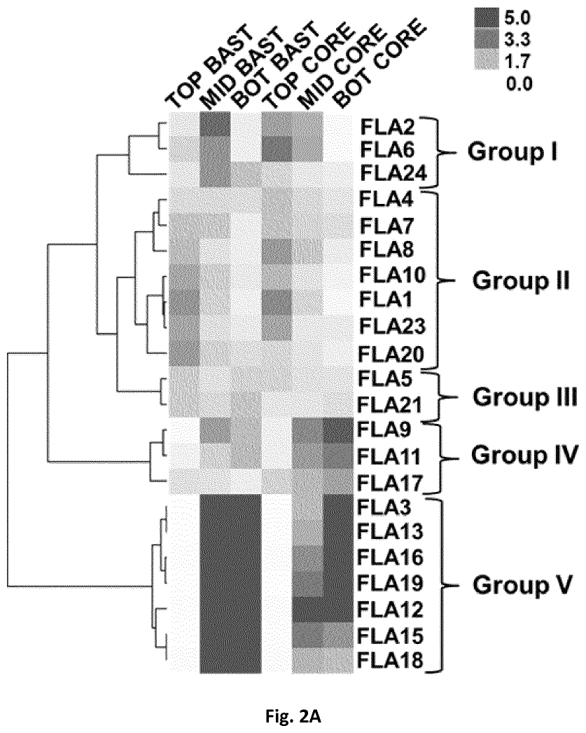

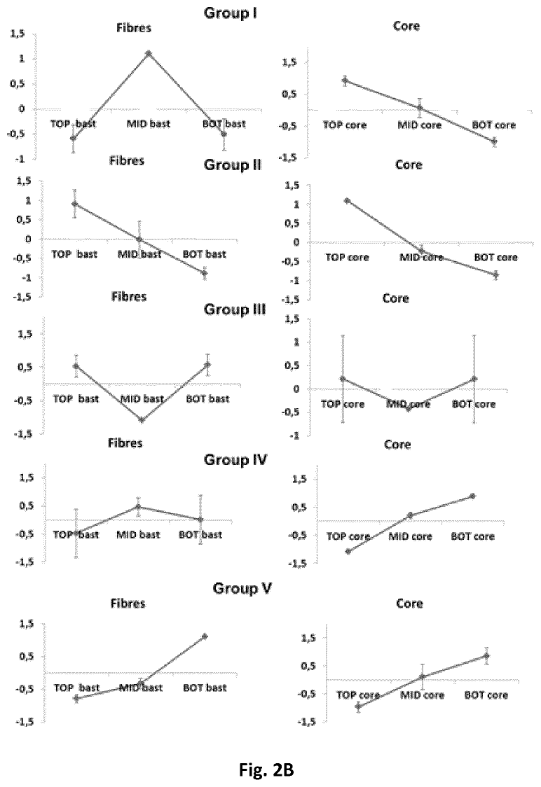

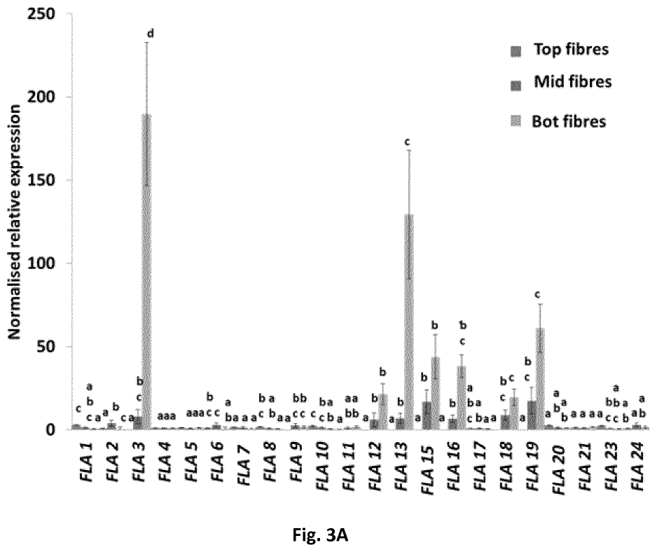

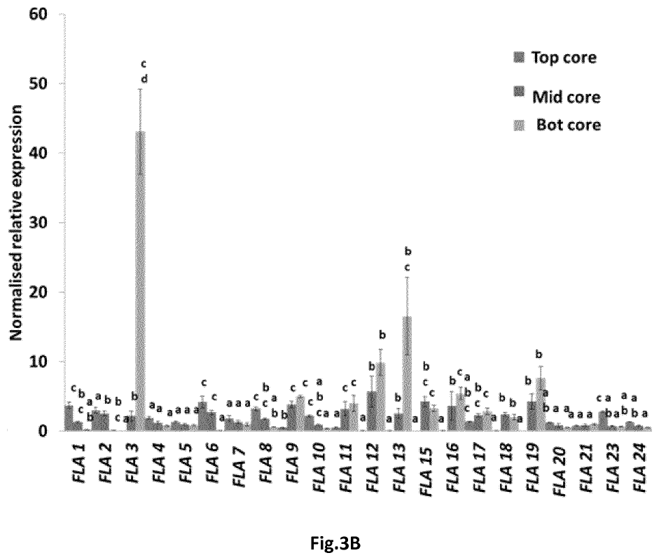

Of the 23 CsaFLAs identified, 22 were expressed in the stem tissues (see FIGS. 2 and 3). CsaFLA14 was detected at very low levels in the stem tissues (Ct>32).

In hemp fibres, the heat-map hierarchical clustering shows five major expression trends (FIG. 2A) in two different fibrous parts of the plant, the bast fibres (namely the skin fibres) and the core (the internal part of the stem), at the three different zones (TOP/ASP, MID, BOT/BSP) indicated on FIG. 1.

These five major expression trends are the following:
1) a group of genes (CsaFLA 2-6-24, also SEQ-ID NO:6, SEQ-ID NO:10 and SEQ-ID NO:27, respectively) is upregulated at the middle internode containing the snap point (in the core the expression decreases towards the base of the stem);
2) CsaFLA 1-4-7-8-10-20-23 (also SEQ-ID NO:5, SEQ-ID NO:8, SEQ-ID NO:11, SEQ-ID NO:12, SEQ-ID NO:14, SEQ-ID NO:24, and SEQ-ID NO:26, respectively) are expressed at higher levels in the top and decreased towards the bottom internode;
3) two FLAs, CsaFLA 5 and 21 (also SEQ-ID NO:9 and SEQ-ID NO:25, respectively), are downregulated at the snap point;
4) three genes, CsaFLA 9-11-17 (also SEQ-ID NO:13, SEQ-ID NO:15 and SEQ-ID NO:21, respectively), show a tendency to upregulation at the snap point, although the pattern is less marked with respect to group I (and in the core the expression increased towards the stem base);
5) the last group comprises FLAs upregulated at the bottom (CsaFLA 3-12-13-15-16-18-19, also SEQ-ID NO:7, SEQ-ID NO:16, SEQ-ID NO:17, SEQ-ID NO:19, SEQ-ID NO:20, SEQ-ID NO:22, SEQ-ID NO:23, respectively) (see FIG. 2B).

The genes of groups I to IV do not show the expression pattern that increases as the stem internodes become more lignified (in the BSP part). It is desirable to drive expression during thickening so as not to interfere with elongation of bast fibres.

In contrast, the genes of the group V are expressed in the bast fibres at the zones MID and BOT while not expressed in the TOP part of the plant.

Subsequently, the genes in group V, i.e. CsaFLA3, CsaFLA12, CsaFLA13, CsaFLA15, CsaFLA16, CsaFLA18 and CsaFLA19, (SEQ-ID NO:7, SEQ-ID NO:16, SEQ-ID NO:17, SEQ-ID NO:19, SEQ-ID NO:20, SEQ-ID NO:22, and SEQ-ID NO:23, respectively) are considered as the gene of choice for driving the expression of the heterologous gene, because the expression would take place during fibre thickening and not during fibre elongation (that would have been expressed in the TOP bast fibre). By selecting those genes of group V, there will not therefore be any interference with the elongation phase. These promotors can thus enter in interaction with a cellulose binding modules (CBMs).

Those genes do not even express in the elongation phase (TOP) of the core fibres, as shown by FIG. 2A.

Choice of the cellulose binding modules (CBMs).

In order to expand the properties of the cells, the functional proteins must be attached to the cell wall of the bast fibre, and more particularly in the outer layer of the cell wall, which is the outermost structure found after degumming of bast fibres. When the expression cassettes are designed, it is therefore important to favour the fusion to a protein domain that will favour the binding of the proteins of interest into the outer layer of the bast fibre cell wall.

Among the different CBMs that are known, those recognizing xylan were chosen since xylan is known to be present in the outer cell wall layer of hemp bast fibres (Blake A. W. et al., Planta, 2008, 228, 1-13).

More particularly, the choice of CBM15 and CBM35 has been driven by the fact that they bound xylan. CBM15 binds to all the secondary cell walls and also recognizes the outer cell wall layer in the flax and in the tobacco stem (Szabó L. et al., J. Biol. Chem., 2001, 276 (52), 49061-49065). CBM35 binds specifically the secondary cell walls of the pea stem, and to both primary and secondary cell walls of flax in a manner similar to CBM15 (Bolam D. N. et al., J. Biol. Chem., 2004, 279 (22), 22953-22963).

CBM15 is present in SEQ-ID NO:1 (>xynC Cellvibrio Japonicus S13392). More in particularly, CBM 15 is SEQ-ID NO:2.

CBM35 is present in SEQ-ID NO:3 (>xyn10C Cellvibrio Japonicus NC_010995.1). More in particularly, CBM35 is SEQ-ID NO:4.

The use of such CBM recognizing xylan increases the chance of expressing the gene of interest (for instance, coding for a surface-active proteins, an elastomeric protein, a phosphoprotein or a spheroprotein) into the outer cell wall layer of bast fibres in fibre crops (notably, hemp).

On the other hand, if the CBMs are not used, the recombinant protein can still be expressed in the plant, but not among the surface cells of the plant. In this case, the expression may not end up in the outer layer of the cell wall and may be in the inner layer or elsewhere and interfere with the correct cellulose fibril assembly and final cellulose crystallinity.

Design of the Expression Cassette (Comprising the CBMs) by Polymerase Chain Reaction (PCR)

DNA primers are designed to amplify the promoter of the identified genes, using as template hemp genomic DNA. DNA primers are used to create the expression cassette.

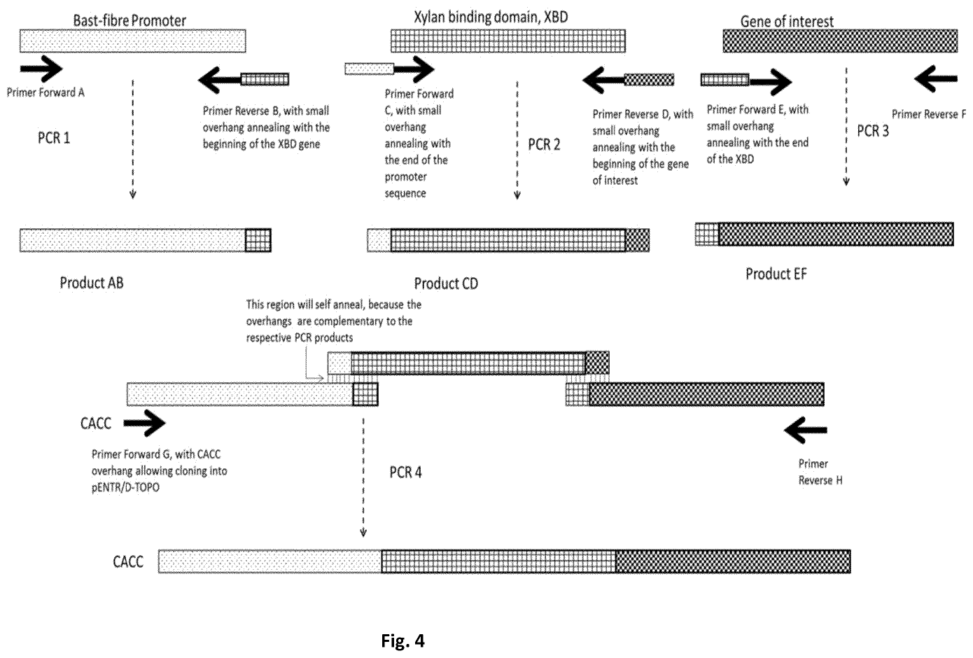

FIG. 4 indicates the PCR process between the bast-fibre promoter, the gene for the cellulose binding domain recognising xylan and the gene of interest.
As the bast-fibre promoter is a double-stranded DNA fragment, a forward primer A and a reverse primer B are needed for achieving the first PCR step. The reverse primer B contains a small overhang adapted for annealing with the beginning of the gene of the cellulose binding domain recognizing xylan. This first PCR step is yielding the product AB.
Similarly, as the gene of the cellulose binding domain recognizing xylan is a double-stranded DNA fragment, a forward primer C and a reverse primer D are needed for achieving the second PCR step. The forward primer C contains a small overhang adapted for annealing with the end of the promoter sequence. The forward primer C contains a small overhang adapted for annealing with the beginning of the gene of interest.
This second PCR step is yielding the product CD.
In a third PCR step, realised with the gene of interest, a forward primer E and a reverse primer F are needed. The forward primer E contains a small overhang adapted for annealing with the end of the gene of the cellulose binding domain recognizing xylan.
This third PCR step is yielding the product EF.
Then, in the course of the denaturation/annealing step inherent to a fourth PCR step, the products Aft CD and EF are coupled together. They have been indeed designed to self-anneal in the course of the PCR. A forward primer G, containing a small overhang with the sequence CACC adapted for cloning the cassette in the pENTR™/D-TOPO® vector is used.
This fourth PCR step is subsequently yielding a cassette which is composed of the bast-fibre promoter fused to the gene coding the gene of interest through the cellulose binding domain recognizing xylan.

This cassette is then cloned in the pENTR™/D-TOPO® vector and recombined into the pEarleyGate 302 vector.

Design of the Expression Cassette (Not Comprising the CBMs) by Polymerase Chain Reaction (PCR)

DNA primers are designed to amplify the promoter of the identified genes, using as template hemp genomic DNA. DNA primers are used to create the expression cassette.

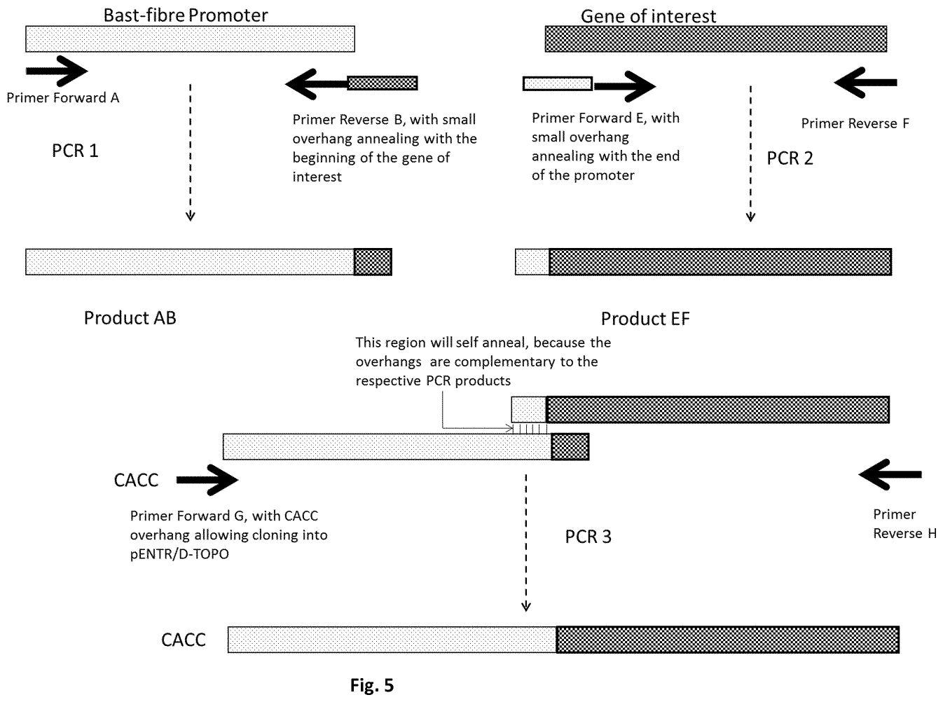

FIG. 5 indicates the PCR process between the bast-fibre promoter and the gene of interest.
As the bast-fibre promoter is a double-stranded DNA fragment, a forward primer A and a reverse primer B are needed for achieving the first PCR step. The reverse primer B contains a small overhang adapted for annealing with the beginning of the gene of interest.
This first PCR step is yielding the product AB.
Similarly, as the gene of interest is a double-stranded DNA fragment, a forward primer E and a reverse primer F are needed for achieving the second PCR step. The forward primer E contains a small overhang adapted for annealing with the end of the promotor sequence.
This second PCR step is yielding the product EF.
Then, in the course of the denaturation/annealing step inherent to a third PCR step, the products AB and EF are coupled together. They have been indeed designed to self-anneal in the course of the PCR.
A forward primer G, containing a small overhang with the sequence CACC adapted for cloning the cassette in the pENTR™/D-TOPO® vector is used.
This third PCR step is subsequently yielding a cassette which is composed of the bast-fibre promoter fused to the gene coding the gene of interest.

This cassette is then cloned in the pENTR™/D-TOPO® vector and recombined into the pEarleyGate 302 vector. Results obtained using the pEarleyGate 103 vector harbouring the constitutive 35S promoter have shown that the cassette comprising CBM35 and RodA can be stably integrated in tobacco regenerants after Agrobacterium transformation.

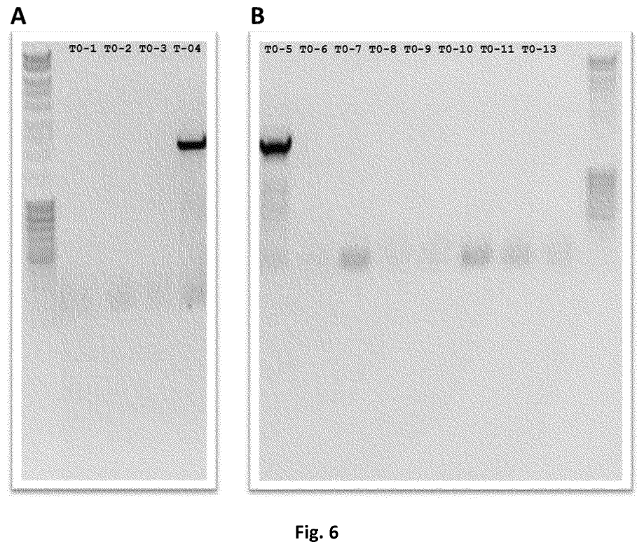

FIG. 6 shows 2 plants with the cassette CBM35-RodA stably integrated in the genome. In fact, PCR has been carried out on DNA extracted from 12 independent regenerants (T0) using the primers Cassette Fwd CACCATGCAGTTGGTACGGTCAGTTTGTTT and Cassette Rev TCACACGTGGTGGTGGTGGTGGTGGCT. The bands correspond to the CBM35-RodA cassette integrated in the tobacco plants. The lines T-04 and T-05 show integration of the transgene.

Choice of the Gene of Interest

The gene of interest which is used in the described process is a gene which codes for a surface-active protein, for an elastomeric protein, a phosphoprotein or a spheroprotein.

The surface-active protein is selected from the group of hydrophobins (they form a hydrophobic coating on the surface of an object), chaplins, rodlins (both chaplin and rodlin proteins are also involved in the hydrophobic properties of surface) and streptofactins (an extracellular hydrophobic peptide).

The elastomeric protein is resilin. Resilin can be found notably in many insects, enabling them to jump or to pivot their wings efficiently. Resilin also increases the resilience of the surface.

The phosphoprotein is casein, which can be used as a flame-retardant.

The spheroprotein is whey protein, which can also be used as a flame retardant.

Antimicrobial peptide can also be targeted via the expression cassette of the present invention.

Experimental Results

Protocol for the amplification of the cassette composed of the promoter, the CBM35 and the hydrophobin as gene of interest

Genomic hemp DNA was extracted from stem tissues (whole internodes) by using a CTAB-based protocol coupled to the NucleoSpin Plant II kit (Macherey-Nagel). Briefly, 500 μl of extraction buffer (2% CTAB, 2.5% PVP-40, 2 M NaCl, 100 mM Tris-HCl pH 8.0, 25 mM EDTA and 10 μl RNase) were added to 100 mg of finely ground sample and the slurry was vortexed vigorously. After an incubation step at 60° C. for 10 min, 20 μl β-ME/ml buffer were added and the samples were further incubated for 20 min at 60° C. Subsequently, 500 μl chloroform/isoamyl alcohol 24:1 were added, the samples were vortexed and centrifuged at RT for 10 min at 10000 g. To the aqueous phase, ⅔ cold isopropanol were added and the DNA was precipitated for 1 h at −20° C. After this stage, the Nucleospin II columns were used to bind the DNA and the manufacturer's instructions were followed to elute genomic DNA.

PCRs were performed using 50 ng DNA and the Q5 Hot Start High-Fidelity 2× Master Mix, following the manufacturer's instructions.

The PCR consisted of a denaturation step at 98° C. for 1 minute, then 30 cycles of denaturation at 98° C. for 10 seconds, annealing at 57° C. for 30 seconds, extension at 72° C. for 1 minute and 30 seconds; after the cycling a final extension at 72° C. for 2 minutes was carried out and then the reactions were kept on a hold at 12° C. The PCR primers to amplify the promoter of FLA16 are

| | |
|---|---|
| CsaFLA16-F1 | SEQ-ID NO: 28 |
| CsaFLA16-R1 | SEQ-ID NO: 29 |
| CsaFLA16-F2 | SEQ-ID NO: 30 |
| CsaFLA16-R2 | SEQ-ID NO: 31 |

Once amplified, the PCR product was reamplified with the primers

| | |
|---|---|
| CsaFLA16-F3 | SEQ-ID NO: 32 |
| CsaFLA16-R3 | SEQ-ID NO: 33 |

The same cycling parameters described above were used.

The CBM domain of SEQ-ID NO:2 was amplified form plasmid pDB1 (Bolam D. N. et al., J. Biol. Chem., 2004, 279 (22), 22953-22963) with primers

| | |
|---|---|
| CBM35 Fwd | SEQ-ID NO: 34 |
| CBM35 Rev-RodA | SEQ-ID NO: 35 |

and the following PCR parameters:
denaturation step at 98° C. for 1 minute, then 30 cycles of denaturation at 98° C. for 10 seconds, annealing at 58° C. for 30 seconds, extension at 72° C. for 30 seconds; after the cycling a final extension at 72° C. for 2 minutes was carried out and then the reactions were kept on a hold at 12° C.

The hydrophobin was amplified from cDNA of Aspergillus nidulans mycelium. The cDNA was obtained as hereafter described. Total RNA from A. nidulans hyphae was extracted from 100 mg of finely pulverized tissue, by using the RNeasy Plant Mini Kit (Qiagen), coupled with the on-column DNaseI digestion. The quality of the extracted RNA was checked by electrophoresis and the concentration measured using a ND-1000 spectrophotometer (NanoDrop). One microgram of extracted RNA was retro-transcribed using the iScript cDNA Synthesis kit (Biorad), following the manufacturer's instructions. The RodA gene (hydrophobin) was amplified with the Q5 Hot Start High-Fidelity 2× Master Mix, by using primers

| | |
|---|---|
| RodA Fwd | SEQ-ID NO: 36 |
| RodA Rev without Stop | SEQ-ID NO: 37 |

and ca. 50 ng of cDNA and the following PCR parameters:
denaturation step at 98° C. for 1 minute, then 30 cycles of denaturation at 98° C. for 10 seconds, annealing/extension at 72° C. for 1 minute; after the cycling a final extension at 72° C. for 2 minutes was carried out and then the reactions were kept on a hold at 12° C.

The promoter, CBM35 and hydrophobin PCR products were purified using the QIAGEN PCR Purification kit and the cassette was created by using as template 2 μl of purified products (between 15-30 ng DNA) in a 20 μl of final volume and the Q5 Hot Start High-Fidelity 2× Master Mix and the following cycling program:

denaturation step at 98° C. for 1 minute, then 30 cycles of denaturation at 98° C. for 10 seconds, annealing at 66° C. (with an increase of 0.2° C./cycle) for 30 seconds, extension at 72° C. for 2 minutes; after the cycling a final extension at 72° C. for 2 minutes was carried out and then the reactions were kept on a hold at 12° C.

The product was PCR purified and recombined into the Gateway vector following the manufacturer's instructions.

Protocol for the Amplification of the Cassette Composed of the Promoter and the Hydrophobin as Gene of Interest

Genomic hemp DNA was extracted from stem tissues (whole internodes) by using a CTAB-based protocol coupled to the NucleoSpin Plant II kit (Macherey-Nagel). Briefly, 500 μl of extraction buffer (2% CTAB, 2.5% PVP-40, 2 M NaCl, 100 mM Tris-HCl pH 8.0, 25 mM EDTA and 10 μl RNase) were added to 100 mg of finely ground sample and the slurry was vortexed vigorously. After an incubation step at 60° C. for 10 min, 20 μl β-ME/ml buffer were added and the samples were further incubated for 20 min at 60° C. Subsequently, 500 μl chloroform/isoamyl alcohol 24:1 were added, the samples were vortexed and centrifuged at RT for 10 min at 10000 g. To the aqueous phase, ⅔ cold isopropanol were added and the DNA was precipitated for 1 h at −20° C. After this stage, the Nucleospin II columns were used to bind the DNA and the manufacturer's instructions were followed to elute genomic DNA.

PCRs were performed using 50 ng DNA and the Q5 Hot Start High-Fidelity 2× Master Mix, following the manufacturer's instructions.

The PCR consisted of a denaturation step at 98° C. for 1 minute, then 30 cycles of denaturation at 98° C. for 10 seconds, annealing at 57° C. for 30 seconds, extension at 72° C. for 1 minute and 30 seconds; after the cycling a final extension at 72° C. for 2 minutes was carried out and then the reactions were kept on a hold at 12° C. The PCR primers to amplify the promoter of FLA16 are indicated below:

| | |
|---|---|
| CsaFLA16-F1 | SEQ-ID NO: 28 |
| CsaFLA16-R1 | SEQ-ID NO: 29 |
| CsaFLA16-F2 | SEQ-ID NO: 30 |
| CsaFLA16-R2 | SEQ-ID NO: 31 |

Once amplified, the PCR product was reamplified with the primers

| | |
|---|---|
| CsaFLA16-F3 | SEQ-ID NO: 32 |
| CsaFLA16-R3 | SEQ-ID NO: 33 |

The same cycling parameters described above were used.

The hydrophobin was amplified from cDNA of Aspergillus nidulans mycelium. The cDNA was obtained as hereafter described. Total RNA from A. nidulans hyphae was extracted from 100 mg of finely pulverized tissue, by using the RNeasy Plant Mini Kit (Qiagen), coupled with the on-column DNaseI digestion. The quality of the extracted RNA was checked by electrophoresis and the concentration measured using a ND-1000 spectrophotometer (NanoDrop). One microgram of extracted RNA was retro-transcribed using the iScript cDNA Synthesis kit (Biorad), following the manufacturer's instructions. The RodA gene (hydrophobin) was amplified with the Q5 Hot Start High-Fidelity 2× Master Mix, by using primers

| | |
|---|---|
| RodA Fwd | SEQ-ID NO: 36 |
| RodA Rev without Stop | SEQ-ID NO: 37 |

and ca. 50 ng of cDNA and the following PCR parameters:
denaturation step at 98° C. for 1 minute, then 30 cycles of denaturation at 98° C. for 10 seconds, annealing/extension at 72° C. for 1 minute; after the cycling a final extension at 72° C. for 2 minutes was carried out and then the reactions were kept on a hold at 12° C.

The promoter and hydrophobin PCR products were purified using the Qiagen PCR Purification kit and the cassette was created by using as template 2 μl of purified products (between 15-30 ng DNA) in a 20 μl of final volume and the Q5 Hot Start High-Fidelity 2× Master Mix and the following cycling program:
denaturation step at 98° C. for 1 minute, then 30 cycles of denaturation at 98° C. for 10 seconds, annealing at 66° C. (with an increase of 0.2° C./cycle) for 30 seconds, extension at 72° C. for 2 minutes; after the cycling a final extension at 72° C. for 2 minutes was carried out and then the reactions were kept on a hold at 12° C.

The product was PCR purified and recombined into the Gateway vector following the manufacturer's instructions.

Protocol for tobacco transformation after agroinfiltration (based on Sparkes I. A., et al., Nat. Protoc., 2006, 1 (4), 2019-2025). Agrobacterium tumefaciens GV3101-pMP90 was grown overnight at 28ig and 130 rpm, centrifuged at 1000 g for 10 minutes, then washed with infiltration buffer (20 mM MES, 20 mM MgSO₄). The bacteria were resuspended in infiltration buffer supplemented with 150 mg/L acetosyringone to a final OD 600 nm of 0.1 and left at room temperature without agitation for 3 hours. Fully expanded leaves of 4-weeks-old tobacco (Nicotiana tabacum var. Black Sea Samsun) were infiltrated with the bacteria and left in the incubator (6 h light 25cteria and left, 60% humidity) for 7 days. The infiltrated leaves were then sampled and surface sterilized under a hood, by gently shaking them for 8 minutes in a solution 1:1 of sodium hypoclorite 14%, sterile water and 0.01% polysorbate 20 (Tween® 20). The leaves were then washed 3-4 times in sterile water, cut to small pieces and put with the abaxial side on the shooting medium (prepared as in Sparkes I. A. et al. 2006, with the exception of the antibiotic concentrations to kill Agrobacterium which were doubled and the presence of BASTA at 5 mg/L). The regenerants (T0) were then put on rooting medium (prepared as in Sparkes I. A. et al. 2006, with the exception of the antibiotic concentration to kill Agrobacterium which were doubled and the presence of BASTA at 5 mg/L). When the root system was well developed, the plants were transferred to pots in an incubator.

The PCR on the leaves of the regenerants was performed with the Phire Plant Direct PCR kit (Thermo Scientific™) using the following PCR cycling conditions: 98° C. for 1 minute, then 30 cycles of denaturation at 98° C. for 10 seconds, annealing at 58° C. for 30 seconds, extension at 72° C. for 3 minutes; after the cycling a final extension at 72° C. for 2 minutes was carried out and then the reactions were kept on a hold at 12° C.

1-20. (canceled)

21. A genetic engineering process of a fibrous plant comprising bast fibres, said process comprising the following steps:
a) identification of one bast fibre promoter;
b) amplification of the bast fibre promoter; and
c) preparing an expression cassette by fusing the bast fibre promoter with at least one gene coding for a first protein through a protein domain, the protein of the protein domain being a second protein different from the first protein,
wherein the step (a) is performed in a below snap-point part of the stem of the fibrous plant.

22. The genetic engineering process according to claim 21, wherein the step (a) is the identification of one bast fibre promoter driving the expression of a transgene during the thickening phase of the bast fibres.

23. The genetic engineering process according to claim 21, wherein the protein domain is a cellulose-binding domain recognising xylan, the protein domain is SEQ ID NO:2 or SEQ ID NO:4.

24. The genetic engineering process according to claim 21, wherein the bast fibre promoter is one promoter selected from the group of SEQ-ID NO:7, SEQ-ID NO:16, SEQ-ID NO:17, SEQ-ID NO:19, SEQ-ID NO:20, SEQ-ID NO:22, and SEQ-ID NO:23.

25. The genetic engineering process according to claim 21, wherein the process further comprises the following step:
d) cloning the expression cassette in a first vector, the pENTR™/D-TOPO® vector.

26. The genetic engineering process according to claim 25, wherein the process further comprises the following step:
e) recombining the first vector, the pENTR™/D-TOPO® vector, into a second vector, the pEarleyGate 302 vector.

27. The genetic engineering process according to claim 21, wherein the step (a) is performed by formation of complementary deoxyribonucleic acid libraries and subsequent high-throughput sequencing, the formation and sequencing being performed by using quantitative reverse transcription polymerase chain reaction (RT-qPCR).

28. The genetic engineering process according to claim 21, wherein the step (b) is performed by means of polymerase chain reaction.

29. The genetic engineering process according to claim 21, wherein the first protein is a surface-active protein, an elastomeric protein, a phosphoprotein or a spheroprotein.

30. The genetic engineering process according to claim 29, wherein the surface-active protein is selected from the group of hydrophobins, chaplins, rodlins, and streptofactins.

31. The genetic engineering process according to claim 29, wherein the elastomeric protein is resilin.

32. The genetic engineering process according to claim 29, wherein the phosphoprotein is casein.

33. The genetic engineering process according to claim 29, wherein the spheroprotein is whey protein.

34. The genetic engineering process according to claim 21, wherein the plant is selected from the group of flax, hemp, jute, kenaf, ramie and nettle.

35. The genetic engineering process according to claim 21, wherein the process is performed by Agrobacterium tumefaciens GV3101.

36. An expression cassette comprising a bast fibre promoter identified in a below snap-point part of the stem of a fibrous plant and at least one gene coding for a protein, wherein the bast fibre promoter is fused to a protein domain fused itself to the at least one gene coding for a first protein, the protein of the protein domain being a second protein different from the first protein.

37. The expression cassette according to claim 36, wherein the protein domain is a cellulose-binding domain recognising xylan, the protein domain is SEQ-ID NO:2 or SEQ-ID NO:4.

38. A plant comprising within its genetic code at least one expression cassette comprising a bast fibre promoter identified in a below snap-point part of the stem of a fibrous plant and at least one gene coding for a protein, wherein the bast fibre promoter is fused to a protein domain fused itself to the at least one gene coding for a first protein, the protein of the protein domain being a second protein different from the first protein.

39. The plant according to claim 38, wherein the plant is selected from the group of flax, hemp, jute, kenaf, ramie and nettle.

40. A transgenic seed of a plant, the seed comprising at least one expression cassette comprising a bast fibre promoter identified in a below snap-point part of the stem of a fibrous plant and at least one gene coding for a protein, wherein the bast fibre promoter is fused to a protein domain fused itself to the at least one gene coding for a first protein, the protein of the protein domain being a second protein different from the first protein.


SEQUENCE LISTING (25 sequences)

SEQ ID NO: 1 - DNA, 1716 bp, Cybister japonicus
atgattaaccacaacaagactcccaacatcctcgccaaggtatttaaacgtacctgcggcctggtgagtactggtgcagccctggcgatcttatcccaggctgcgtccgccgcttgtacctacaccatcgatagcgaatggtccaccggctttaccgccaatatcaccctcaaaaacgataccggtgccgccatcaataactggaacgtgaattggcaatactccagcaatcgcatgaccagcggctggaatgccaacttctccggcaccaacccctacaacgccaccaacatgagctggaacggcagcatcgcgccaggacaatccatctccttcggcctccagggcgaaaaaaatggcagcaccgccgagcgaccaaccgtcaccggcgccgcttgtaacagtgcaaccaccagctctgtggcttccagctcttcaacacccaccaccagttcatcttctgcatccagtgtggcctccgcactgctgttgcaagaagcacaagccggtttctgccgtgtggatggcaccatcgataataaccacaccggctttaccggcagtggctttgccaacaccaacaatgcccagggggcagcggtagtctgggcgatagatgctaccagcagtggccgtcgcaccctgactatccgctatgccaatggtggaaccgccaatcgcaatggctcactggtgattaacggcggcagcaacggtaactatacggtgagtttgcccacgaccggcgcctggaccacctggcaaaccgcaactatcgatgtggatttggtacagggcaataatattgtgcagttgtctgcaacgacagccgaaggcttacccaatatagattcgttaagtgttgtgggtggtacggtcagggcgggtaattgcggcagtgtgagcagcagcagttccgtgcaatcgtcatcctcctcatcaagtaccccatcacaaacctgtgagttaaaagcccccttgcgctggacctcgaccggccccttgatcagccctaaaaatccaggctggatatccatcaaggatccgtccatcgtgaaatacaacgacacctaccatgtgtatgcaacctactatgacactgcctaccgatccatgtataccagctttaccgactggaacacggcacagcaggcaccgcatatttccatgaacggcagccgtgtcggcaataccgttgcaccacaggtgttttatttccgcccgcataacaaatggtatttgattacccagtgggcaggtgcttatgcaaccactgacgatatccgtaaccccaactggtcagccaaacaaaaactgctacagggtgagcctaatggcgcgctggatttctgggtaatttgtaacgatacccattgttatttgtatttctcgcgcgacgatggtgtgttgtatgtatccaagacaacccttgccaacttcccgaatttctcaggctattcgattgtgatggaagatcaccgcggcaatggcaacagctatctgtttgaagcagccaatgtgtacaagctggatggacaaaaccgatatctgttaatggtggaagcctatatcagcggccgcgctttttccgctcctggacagcgaccagcctggatggcccatggacccttggctgacacagaagccaatccctttgccgggatgatgttttgtttcacgatggcttcctcattaaaggtttacacctgttattaa

SEQ ID NO: 2 - DNA, 1821 bp, Cybister japonicus
atgaaaaagatccagcaactcctcatgctctccctgattagctcaacactaattgcctgtggcggcggtggagggggcggttcaacaccaaccaccagcagctcaccccaatcctctagtccagcatcgacgccatccagtgcttcatcctcatcgatcatatcctcctcatcgctatcatcatcgctgtcttcctcatcactatcctcgtcatcgctgtcttcttcatcggcgagcagtgttagcagctccagtgtcgctgccagcgagggcaatgttgttatagaggtggacatggcaaatggctggagaggcaacgcatcaggcagtaccagccattccggtattacctacagtgccgatggcgttacctttgccgcactgggtgatggcgtgggcgctgtttttgatattgcccgaccaaccacactggaagatgctgtgatagcaatggttgttaatgtcagcgctgaatttaaggccagtgaagccaacttgcagatatttgcccagttaaaagaagactggtcaaagggcgaatgggattgtctggcggccagcagcgaactcactgcggatactgacctaaccctgacctgcaccattgatgaagacgacgataaattcaaccaaacggcgcgcgatgtacaagtcggtatccaggccaagggaacacccgccggaactatcaccattaaaagcgtcaccattacactcgcacaggaagcctattcagccaatgtcgatcacctgcgcgacctggcccccagcgatttccccattggcgtcgccgtgtccaataccgactccgccacttacaacctgctcaccaacagcagagagcaggctgtggttaaaaagcacttcaatcatttaactgccggtaacatcatgaagatgagttacatgcaacctaccgagggcaattttaacttcaccaacgccgacgcctttgtggattgggccactgaaaataatatgacggtgcacggccacgccctggtatggcattccgattaccaggttcccaactttatgaaaaactgggcaggcagtgcagaagactttttagcggccttggacacacatatcaccaccattgtcgatcactacgaagccaagggtaacctcgtcagttgggacgtcgttaacgaagccatcgacgacaacagtccggcaaacttccgcacgacggattctgcgttttatgtgaagagcggaaacagctctgtctatattgagcgcgccttccagaccgcgcgcgcggcagatcccgctgtgatcctctactacaacgactacaacattgagcagaacaatgccaagaccaccaaaatggtcgatatggtcaaggacttccaggcgcgcagtatcccgattgacggtgtgggcttccagatgcatgtctgtatgaactatccatccatcgccaacatttctgccgcgatgaagaaagtggtcgaccttggcttgctggtaaaaattaccgaactggatgttgccgtcaaccagccccattgcgatgcgtatccggccaacaaaatcaatccgctgaccgaagcggcgcaattggcccagaaaaaacgctactgcgacgtagtgaaggcctatctggatacagtcccggtgaatcagcgcggcggtatcagcgtctggggaactaccgatgccaacacctggcttgatggcctgtacagagaacagtttgaggatgaaaaaatttcctggcccttgctgttcgacaacaactacaacgacaaacctgcactgcgcggttttgccgatgcgttaatcggtacgcaatgtactaatacacattga

SEQ ID NO: 3 - DNA, 1263 bp, Cannabis Sativa L.
atgcagctccgtccggccttaaccgccgggacactggtcctagctatggccgtcatcttcatcttctccggcaccggtgtcgatgctcacaacatcacgaagattctcgctaagcatccggagttttcttcattcaaccattacctaaccctaactcatctcgcctcagaaatcaaccagcggacgacgattactgtctgcgccgttgataacgccgccatgggcgatattctctcgaagcatccgaatatctatactgtaaagaatattctctcgattcatgttcttctcgattatttcggtgccaagaagcttcaccagatcactaacggtactgctcttgctgctactatgtttcaagctaccggttccgctcctggatctactggattcgttaatatcacagatctacacggtggaaaggttggattcgctcctgaagataacaacggagcttttgctgcgcattttgtgaaatctgttgaggaaattccttacaatatatcggtgattcagatcagcggtgttttgccttcggctgcggctgcggcgccgactccagctccggctgagattaatattaccggcattatgtcggctcacggctgtaaagccttctctgatgctttacttgctaacgacgctatggaaatttacgaagatgccctcgctggtggtttaactgtgttttgtcctttggatgatgcgttcaaagcttttttgcctaaattcaagaatctaaccaaatcagggaagaattcgttgttagagtaccacggagtacctgtttaccaatccatgtcgatgttgaaatcaaacaacggactcatgaacactctcgctacagacggagctagcaagttcgatttcacagtccagaacgacggagagcaagtgactctcaaaacgaagctcgtcacggccaaaatcaccggcacactcatcgacgaacaaccagtagtgatctacactatcgataaggtattaatgcctaaggagcttttcaagcacgagaaagcagagactccagctccagctccggcaccggaaaaggcagcagacgcacctaaatcttctaagaagaaaggcaagaaagcggcgccgtcacctgacgacgacgccgatgccgacgctccagcaccagacgatgatgacgtcgcagatcagactgctgatgataacggcgccgttagatttgacggtaacagatttagcttcttcgctcttatcgctactgcatggctaggattttcagttctgtaa

SEQ ID NO: 4 - DNA, 1167 bp, Cannabis Sativa L.
atgcctcatcatcaccaaaaatttctcctcctcttcttcttcttcctcgtggcaaccacatcccacgcccacaacatcacgaaaatcctagccaagcaccccgaattgtcgaccttcaaccagtacctgagccgaactcggctggcggccgacatcaaccgccggctgaccatcacagtcctggcggtggacaatgcaggaatgtcatctctcatctcaaagggctattctctctacactattcgcaacatcctctcactccacgtccttgttgactattttggtgcgaaaaagctccaccagatcagcaagggctccaccttgacttccagtgtcttccaagctagtggtgccgccccaggcacctccggcttcgtcaacatcactgacctcaagggtggtaaggtcgtatttggtgttgaggacaatgatggtcatctcacttctcactttgtcaagtccatcaaggagattccatacaatatctccgtcattgaaatcagtcaggtgttgagttcggccgaagccgaagcgccgacttcaggaccaagcgagttgaacgtaacgactatattgtcaaagcaaggctgcaaatctttcgccgacttgttaatagccactggagccgacgccacttaccagtccaacactgagtctggcttaacggtgttttgtccaacagacggtgttgtcaaaggtttcatgccaaagtacaaaaacttaacgactgccaaaaaagtatcgttactgttatatcacggcatccccgtttaccaatccatccagatgcttaagcaaaataacggagtcgttaacacgctggctaccgaccgtgccaataaatatgatttcacggtgcaaactgacggtgaagacttgacgttagagactacggtggtaacgtcgaaggttactgggacgttgattgacaaggagccgttagccatttataagctgaataaggttttgttgcctaaggagctgtataagccgactgaagccacttcgcccaaatccagtagcgatgactccgacgacgaagaagccgatgccccagagggtgattctgatgatcagacggctgacgataatggtgccgttggaataaacggtgggagaatggctgtggtctttttgagcttgtgcgtcggatttttgcttatgtga

SEQ ID NO: 5 - DNA, 825 bp, Cannabis Sativa L.
atgaaaaaacaaggcctcatctccctttccttcatttccatctttttctccatagccctagctcaatctccaacccaagctccaacccaaaccctagcccaagcaccaattacatcaccaacaccatccgaagcgccactagtccaaccgccggccctggcaaaccctacaaacgccaccgagatcctcgaaaaagtcgggggattcagtgtcttcgtccacctcctcaaaacaacatcagagaatattcaaatcgaaaaccaactcaaatacatctcaaacagcttgacaatcttagccccatcaaacaaagcattttccaatctcaaaccaaacactctaaactcactcaccacaaaagaaaaactccaactcatacaaaaccacatcattccttccttcataccaatccaaaatttccaaaccctaataaaccctgtacggacacaagcaaattattctcttaatattttggtcgaaggtagttgggttaacatatccacaggagttgttaatgcaacaattaatgcaacaatttatgaggataatcagttagctatttacaaggtcgataaggtacttcttcctcttagaattttccgtgtaaaaccgcgaaaaaaggcggtgggtgcaccggcaccggctcccatatcaagtatggttgttaagcctgatgagtttccaacatcttcgttgattgctcctgctttggctgctttgcttaaagatgcttctgctgtaagtggtgccctttgtcttagtggaaatgggattttgagctttggaattgctgtggtttacgtggtactactttctcttttttag

SEQ ID NO: 6 - DNA, 1439 bp, Cannabis Sativa L.
atggcgatggaagcagacttacccatttcccattttacccctacaccatccatactcttcctcctctttctcctctcttccgccatagccaccgctcccaccgccgctctcaacatcaccaacctcctctcctccttcccagacttctccgacttcgcttcactcctctcctcctcatcatcttcccttgccgccgatctctcccgccgatcctccataactctcctcgccgtcccaaacacatacctctccgcttcatcagatctcacgcgccgcttatcttcatcctcacgcgctgatgtcgtccgataccacatactcctcgaatacctctccccttccgatctccgccggatctcttcctcaggtaagcttgtcaccacgctccttcaaaccaccggacgcgccaccaacaacttcggttccgtcaacatcacccgcgatccaatcaccggaattgtttcagtccgatctccggcaccttattcaccctcgaatgctaccattcttaaccctatcaaaaccctaccgtacaatgtgagtgtcttcgctgttaattctcttctcgttccgtacggattcgatctaatggcttccgagactcgaccgccattaggtttgaatatcactaaggctttaatcgacggtcataacttcaacgttgcggcttcgatgttagcagcttccggcgtagtagaggaatttgaagccgacgaaggtggtgccggaataaccctatttgtaccagtggatactgctttcgctcaacttccgtcaaatgttgagcttcaatcattaccggccgataagaaagcagtggtgttgaaattccatgttcttcattcttattatcctttgggttcgcttgaatcaattgtgaacccagttcaacccacattagctacggaagacatgggcgcaggaagcttcactctcaacatttctagggttaatggatcggtggcaatcaatacaggtatcgttcaagtgtcggtgacccaaacagtttttgatcaaaacccagtcgccatttttggggtttctggggttttattacccaaagagatttttgggaagaatccaataacgacgacgactcccaaaccaggaacaccctttgcttcgggtacggctcagccacctgatattgctttctcgccggagtttacgcctggattagatggccctccttcacacctctcatctccgccgggtttaggtcaagatataagatcgaaagcagaagcagcagccgccattaatgggttccatttctataccttgatccttgctctgttgtgtatagccttatctctttaaatggtaaatggtatgagatataatttattattttggttggtaattagattttattttttttaatttatttatttttgtgttttttatttttttttttacacgggaaattgaatttataa

SEQ ID NO: 7 - DNA, 888 bp, Cannabis Sativa L.
atggcaatggccctctgtctctacctcccagctctcctactcttcttcccaacccttctctcagcttcttcttcttctccttccactacattatcaccgtcgcaatctccaccatctccttctccaccgccaccgccgccttctccttcactcccatccccatctgagtcaaccccaacatctcctcaatcaccacaacattcatcccaccggaggagacaccgaagatcccggcgaaggcaacaacaacctccttcgacagcggagacgcctcagcaattcaacaacataatcgatgccttgattggctccggagacttcagcaattgggtcaatgtcatttcaaacgccgttctccctcttagcgcaactctcttcgtcccagaaaatgaagctgtaactgccccgacgatcgccagtcccggtccggaagacccattgattttcccttaccatgtggtaccacagaggctctcttttgccgaactacttctcttccagaccaacactcgccttcccacgctcctccctgggaagtctatcatcatcaccaacaactcccggatcaacttcaccatcgacggctctctgatcactcagccggatatttactccaccggcaacattgccgttcacggcgttggctctgtctttaattactcggtcttcggtgatggtttagaccttctgcccaaatcctcgaacccagagccgaatcagtcgcaagttcgccggccgccaacggtggatcacccgaacggagagacgtatggttcgagctcggactccgcgccgccgtgcctgtgcattgaattgccggttgttttcttagtgttctgtggggttttgatgttcaagattcagagaaatggcggccatggacggtga

SEQ ID NO: 8 - DNA, 723 bp, Cannabis Sativa L.
atggctttcacacctctctctatcctcctcctgactctcatcaccatcttttcccaccaaatttcagcccaagccccgggccccgccccggccggccccctaaacttcaccgcaatcctcgaaaaaggtggccaatacaccaccttcctccgccttctctccgacagccaagtattaagccaaatcgtaaaccaactcaacacctcctccgaaggactaaccgtcttagccccaaccgacaatgccttcaacaacctcaaggccggaaccctaaacggcctctcccgtgaagaccaagttaacttaatcctcttccacgttctccccaagtactacgccctatcagaactcctcaccgtatccaaccccgtcagaactcagttctctgctgatggccttaacttcaccggtcaaggccgtcaagtcaacgtcaccagcggaatggtggagactcaggtcaacaatgctctgaggatgcaatccccattggctgtttaccagatcgacgatgttttgttgccaccaagtctttttggagctaagccaccggcctctgctcctcctccggccaagactccggcatccaaggacgacggagataagaccaagcctaaggcatccggaccatcgtccgatgatagtactggggattcgagtaatacacgtgtagggttgggattgtttgttgggatgggtattgcttgcatggctgttcttttttga

SEQ ID NO: 9 - DNA, 789 bp, Cannabis Sativa L.
atggcgtacaagtatgttttcattgccagcagtatgctgctgcttctgtgctcatcagcaatggctcaaaaagctgcttcaccacctcccctaatcctaactccaactccggcaccagccccagctcctgattacgtgaatctgaccgaattacttactgtggccggtccattctccaccttccttgactaccttgtatccaccaaagtgattgaaaccttgcaaaaccaagccaacaacactgaagaaggtgtaaccatctttgtcccaaaagacagtgctttcgcatcccttaagcagaagccctctttgaaaaacatcaccaatgatcagctcaagtcactgttgctgttccacgctttgccacattactacagcttagctgagttcaccaatcttagtcaaagcagcccaatcaacaccatggctggtggtccattcactttgaacttcactgatgtttcagggactgtccacatcggctcgggttggaccaacacaaaggtcagcagcagtgttcattcaactgaccctgttgccctttaccaggttgataaagttctcctccctgaggccatttttggaactgatatccctccaaccccagctccagcaccatccccagatattgctcctgttgcagatgcgccaggagatgagggaactgatgccaagtccccatcatcttctacttcacaagggaagtctgcttctcacagggtcaacagcttgggagttttgtgccaaatggttttggctgtttcaggtgggctggccttgttcttgtga

SEQ ID NO: 10 - DNA, 1410 bp, Cannabis Sativa L.
atgggtactcatatccgtggcgaccaaaagcttgtctttttctcattcttcttcatcacattcgccggaatatgcttctcattgccggaaaatgtcaaccccaggtcattgttctcttcctccaacgcttccacatcatcgggtcagatcaactccaactcggtcctggtcgctctcctagactcgcattacactgagctcgctgagctagtcgagaaggctctgcttctccagacacttgaggaagtcgtcggcgccgccaacgttaccattttcgctccacgcaacgaagccctcgagcgaggacttgacccggagttcaagctcttcttgctcgaacctggtaatctcaaatcgcttcaaaagcttatcttgtctcacatcgtaccgacccggatcggatccaacgatttacccaagaaacccgactctgctcaccacagaactctttcccacgaacacatccacctagaaaagcaagattccggtgagtggactgtggatctcgctcgtctcactcatcccgactcggtgacccgacccgatggtgtgatccacgggatcgaaagggttttgattcctcgctcagtagaagacgatttcaaccgtcgacgtagtctacgaacgatcacagccgttaaaccagaaggggctccagtggttgacccgagaacaaaccggttgaagaaaccagctccacctaccaaacccggttcagaaccggctctaccgatctacaacgcaatggctccgggtccatctctagctccagccccagcacccggacccggtggccctcaccaccatttcgatggggagagtcaagtcaaggactttatccaaaccctccttcattacggcggttacaatgaaatggctgacattttggttaacttaacttcgttagccaccgaaatgggtcgattggtttcagagggttacgtactgactgtgctggcaccaaacgatgaagccatggctaagcttactactgaccagctcagtgagccaggagcaccggagcagataatgtactatcacctcataccggagtaccaaactgaagagagtatgtacaactcagttcgccggttcgggaaggtgaaatacgacacacttcgattaccacacaaggttttggcccaagaagctgatgggtcggttcggttcgggcatggggagggttcggcttatttgttcgacccagatatctacaccgacggtcggatttcggttcagggaatcgatggtgttctgttcccgcctgaggaggttgaatccaaacccgtttcccaaacggttcaacccgccaaaattgtagccaagcccaaaagaggaaaattgctagaagtaacatgtcaaatgcttagagtctttggaaaggattcgcaatacccgacatgccactaa

SEQ ID NO: 11 - DNA, 828 bp, Cannabis Sativa L.
atgcaaaataaccaaaaacacaaaccaataaatcctaacaacaaaaactcactacaccaaaacatgaacacatcaaaaatgctcatcaacactccctcacttgttctcattctcctcctcattttcatcacaaaatcttcagctcaaaccgcggccccggctctcccacccggtcccccgccctcgacagacatctacaaaatcctaacaaaagccgggcaattcaccgtcctaattcgcctactcaagagcacacaagttggtaaccaaatcaataaccaactcggaagcacaaacagtgaactaaccatgtttgccccgtccgatagcgccttttcgaacctcaaaaccggaaccctaaacggcctaaccgaccaacaaaaagtccaactccttcaattccacttagtcccatcattcatttcaataaccaatttccaaacaatgagcaaccctgttcaaactcaagccagtgacacttatgagtaccctcttaacataacaacctcaggaagccaagtcaacataacaactggaatagtcaacacaacaatttcaggaacagtttattctgataaccaattggctgtttaccaagttgataaggttcttcaaccacttgggatttttgctccaaggcctctcccaccagctcctgccccagctccacctaaggctagtaagaaaaaagctactgatgctgataccactgggcctgccacgtcggatgacagctcggatgccattggtggtagtaatggaggtttgggcaaggtggtattgtctttgtctatgtctatgtttgttgctgccatttttaagttgtga

SEQ ID NO: 12 - DNA, 1347 bp, Cannabis Sativa L.
atgcaaagctttagctgccattattcgagtatacaaaacaccgttcgatcaacaatttcagttccacaccccaaaatggtcgcactaaaccattatctcatcttctgtcttctattcgccctaaacgccgtcgtttcagcccacaacatcacagaaatcctctccggattcccagagtacagtgattacaataactttttatcccaaacaaagctcagcgatgaaatcaatagccgccaaacactaactgtccttgtcctgagtaacggagctctttcctcccttacagccaaacaccctctctccgttatcaagaaagcccttagccttcacgtcgttctggactacttcgacccgaaaaagctccaccagatctctcagggaagcaccttatccactactctttaccagacttccggaaatgcccctggaaatcttggcttcgtcaacatcaccgaccttaagggcggcaaagttggcttcggttctgcagctcccggctccaagctcgactccacctacgttaagccagtcaagcagattccgtacaacatttcgatccttgagatcagcgcgccaatcattgcgccagggatcttgacggctccggctccatcggcttcagatgctaatataacggcattgcttgagaaagctgggtgtaagacatttgcttcgttgatcgtttcaagcggtgtgattaagacttatatgacggcggtcgagaaaggtttgactctctttgcaccgaacgatgaggctttcaaggctgctggagttccggatctgagcaaactcaccaacgctgagctagtttcggtcttactatatcattccctggctggttactctcccaagggaacgttgaagaccacgagcagcccgatcaaaaccctggctaccaacggggccggaaaatacgaactgactgtatcaactgccggtgacgccgttactctccacacaggagtcgactcttccagacttgccgacacggtaactgactcaaccccacttgtcatattcactgtcgacaatgtcctacttccagcagagttgttcggtaaatcgccatcgcctgcaccagctccagaaccagtgagttctccttctccgtcaccggcacttgctccgactccgggaccagcgaccgaagccccaacccctctcggcgcttccccaccggcaccgccaatggaaactcctggaggagcaccggcgaactctcccgaagctgatgctgaaaacagtaccgcaaaaggagctggtcacatgcacgcgcctgcattgctgactgcacttttcactatctctgccactgtcaccttctcaatcttcttgtcctaa

SEQ ID NO: 13 - DNA, 756 bp, Cannabis Sativa L.
atggagaaaatcacaagattagctctactaataaccacagccattttcctcctttgcaaacctatctcaggccaaagtccggcgaagtcaccggcgccaggaggccctgttgacataatagctgttcttaaaaaagcagggcaatacaccacattcatcaagcttctaaagggtacccaagtatctgaccaaatcaattcccaacttagtggttcaagtcagggcattacagtctttgcacccaccgactcagccttctctagcctcaagacaggcactctcaactccttgaccagtgaacaacaacttcacctagttcagtaccatgtcctcccagccttttataccatctcccaattccaaactgtctccaacccaattcacacccaagctggtaatagcgaaaatggccagtacccacttaatgtaacaacctcgactagtaaccaggtcaacattaccactggtgtggtcaatgctaccgtcagtaataccgtctatactgatggccagctttcagtattcgaagtcgaccaagttttgcttcctttagatatttttggaacggctacagctccggcaccggcgcccgtggctgattctaagcctgttaagagtgttgaccaagattctgatgatgctccggccaagtctaagggccccgatgatgatgacgattctggtgctttgagcctcaaatcaggctcaattggtgggcttttatttgctggagcagctcttcttgcagtcttttaa

SEQ ID NO: 14 - DNA, 942 bp, Cannabis Sativa L.
atggcgaaaaatcaaattttgttcacctattcttgcgcaatagttttcctcttcctcttccatgattgcgaaaatactttaacattagctcaatcgtcatcacctgcccaatcaccatcgacaacacctaataagaatcatcatccttcatctgattcctcagctcaatctccggatcagccattagtggaagccccaccagcagcgaaatccaggagaaaaggacaacccaccaacataacagaaatcctagagaaagccggcggtttcagcatcttcattcgcctactaacaagcactgacgtcatcagtcccgtcgaaaatgatctcaactcttccaataccgtcaccattttggctccaaccaatgcaggattctcagctcttaagaccggaacactcaacactctcaccccccaacaaaaaacacaattggttcagttccacatcatcccaactttcatatccctacagaatttccaaaccctaacaaaccctgttcacactcaagccactaacactcgcgactacccattaaacattactagtactggcggatcttctgtcaacatatctactggagttgtcaacacaaccatatctggaaccatttattccgataaccaactcgctatttatcgagttgatagtgtcttgcttcctatgaaacttttcgctcctaagaaggtggtgagctcactggcaccgtctccagcacccgcaatggccttaaaacctaaaaagaaatcgacggaatcagctccatcgtctggatcgccctcatcctcctcaaaaccatctttaacatctacatcacccacatcatcatcatcatccaatgatgatgaatcagtcgccacatcagcagatacatctggtgctgaacgaccagttgttgttgttgccacagtctccgttggtcttcttgctttgttcttatga

SEQ ID NO: 15 - DNA, 1002 bp, Cannabis Sativa L.
atgatgaaacaagttatcatcttgtccttcttcattgttttcctctttcactcttcttctactttagctcaatctccagctcactccccaactcagcctcaaaaaccaattcctaaagctcaatcgcctactactaaacccgtccttgctcaaccgccatctcaagcagtactaccggctccttctcaagctccaacccaaaagcctctccctcacaccccaccgcgaaagccaacccctaagccagccccgcctaatgtcactgagatccttgacaaagctggaggcttcagtgtcttcgtccgcctccttaaaaacactcaagtcgtcaatcagatcgaaaatcaactcaacacttccaacagcttaaccattttggccccaaccaacggcggattctctagtctaaaagccggcgctctcaacggccttacccctgagcaaaaagtccaactccttcaataccatatccttccatcctacgttcccctccaaaacttcgaaaccctaaccaaccctgtccgtacacaagccagtaacactgaggattaccctatgaacattacaacggagggaaacttcgtaaacatttctactggaattgttaacgctacactttctggtacagtctactctgataatcaactagccatttatagagttgatactgttcttcttcctttaggtatctttggctcgaaagcgcctactccttctcctactcctgctcctactccagctccactcgcgcatttaaaacctaagaagtcttctactccaacaagtacttcatcatcatcctcagctgctttgccttccaactcatcattggcacctaaagcgcctaaaaagccattaaatttgtcgtcgaccacctcttcctcgatcactcctgtggcggcattggatgaatccggtgttgttgccctaacttcaagtaatggcgtcgtgattggccttgttgtgactggggtcgctgcagttatgagtattttgggttaa

SEQ ID NO: 16 - DNA, 930 bp, Cannabis Sativa L.
atgattaagaagatgactaatcccaaagcctcctcctcttctattctcttcatgctcgttgccattttcttattctcttcattatcatcatccttagctttcaacatcacaaaactccttggccaaaatcctgagcttagtacattcaacaactacttaactcagacaaagcttaacgatgagatcaatcgtcgccaaactatcactgtcctcgttgtcgataattccgccgcagcttccctctccggtaagtcactcgatgtaattaagaaaatcttaagcgttcatgtgattttggattactacgatgttgagaagattaccaagcttactacctcgaaaaagacatctaccgtcactactctcttccaagcctccggctccgccgttgatcaagagggttttctcaaagtcgcgctgatcaacgagggtgagatcgccttcggctctgccgtaaagggtgcctctctaaacgccaagttagtcaaatctgtggccgcgcagccattcaacatctccgttctacagatcactgcgccggtccaagtatcgggcattgagtccagcccatcaccaccatctccaaaggcagctacgccttctgctgctcccaaaagggctcccgccccgtcaaacaagtccggggctgctccttccccatccaaaaattctggggccaatactcccgccaccgcacctagtactgctgatgcaccggttgccgatacgccgacaacagccgcaacttcgcctgcgcctgctgctgctgctgctgacgaacccgttgctgataatgccccggtgagttcggctcctccgcaagctgatgctgcagctgatccaccggttttgtcaaattccggggtggtgaggcaggggatgaagataggtattgtggcggcggcattgatgtcgtggttggttgtttaa

SEQ ID NO: 17 - DNA, 948 bp, Cannabis Sativa L.
atgaagcaaactctcatctctttttcattcctacttctcatcttatctcacaccacaatgagtttaactcatcaagctccagctaaagccccatctgcccacatagccgctactaaaggcgccaccccagccgtggcgcctacaaaacctaaagtagcaacaccaactccaacaacatccccatctcgagccccgacctcatcagaagcacaagctccctcagcagagccactagttgaagctccaccgcgaaaagccaaacccgagccaaccgatgtggttaagattcttgacaaagccggaacctttagtgtcttcatccgcatactcaaaagcacccaagtaatagaacagatcgaaaaccaactcaacacttccaacagcatgacaattctagccccaactaacggcgctttctccgctttaaaacccggaacactcaacttcttaaatgcagaacaaaaggtacaattagtacaataccacatcttaccttctttcatttccatctcgagtttccagactctcagtaaccctgtccgtacacaagctagtaacactgacgagtaccctttaaacatcacaacacaaggtaattgggttaacatatcaactggtgttgtcaatacaactatttctggcacaatctatgccgatagccaactcgctatttataaaatcgacaaggttttgcttcccatggccattttcgctcccactaagcttcataaggcccttgcacctgcgcctgcattgcttgctaagcctacaaaaggcttagcaggtagcggtaaagggtcagattcgtcatctgacccctcatcgtctacttcaacattggatgataacccagtttcttcaatgcatgcctctagtgcttttcgtaatggcatggtcgcaacaattggtgttattaatgtttttggtgcaattgcaattttattttga

SEQ ID NO: 18 - DNA, 987 bp, Cannabis Sativa L.
atgaataccaaacactttgtcacgatcttctcattcctaaccctaatcttcttccatgcaacaactttagccacaaccccaactgctcatgccccatcccaatcaccagcacaagcaccagctaaacccttattagctcaacctccaaagaagtcaaagtcttcttcgggggcgcctgccctagactcagcggcctcttctgctccacccttgtcacaagagccaatagtccaagctccaccccacaaaggccgaagcaggattcccaccgacgtggcaggaatcctcgaaaaagtaggaggctttagtgtcttcaaccgcctccttaaaagcaccgaagtactcactcaaatcgaaaaccaactgagtgcctcaaacagcttaaccatcttagccccaacaaacgatgccttcgcctcttcactcaaacccggaacactcaacaccttaaccaaagaacaaaaaatccaaatgatacaataccatgttttaccaactttcatttctctttcgaatttccaaaccctaagcaaccctgtccgtacacaagctgctaatacgtatgattatcctatgaacattactaccgaaggtaattgggttaacatatcgaccggtattgtcaatgcttctattaccgccactgtgttctctgacgatcagttggctatttatagagtggataaggttttgcttcctctcggtgtttttgcaccgagaccaaagcttcaaccttctccttcaccctctgctcttttggctaagccaacaaaagattcttcatcgaattcatcatcatcatcgagttcttcttcaatgtcctccagggcggatggccctggtggagcaagtgaggataatgatgatgatcagaagacgaataatgcttccagtgcatctgattttactattggagctcgaacgatgtcgtttggagctgtaatagtttccatggtcgcaataaaatacattcttgtgcttttttag

SEQ ID NO: 19 - DNA, 1386 bp, Cannabis Sativa L.
atggatactcatggctatggcgtctccttcttattattctttattctttgttcctttactaactctttcgctgcattgcccaacaacccatctcagaaaagtaattctacttcttccggttctggtcagattaattctaactcggttctggttgcgcttctcgactcgcattatactgagttggccgagttagtcgagaaagcccttttgctacagaccttagaagaggccgtcggtaaacacaacatcaccattttcgccccgagaaacgaagctctagaacgccaactcgaccctgaattcaagcgattcctgctcgaacccgggaacgtaaggtctctccagacactcttaatgttccacattatccccaaacgtatcggctccggcgagtggcccgcttccgattccgttccggttagacacaaaactctttggaacgaccgcgttcatttaacgagcaaaaactccggcgaaaaggttatcgactcggccgagatagtccgtcccgacgacgtagtaagacccgacggtgtaatccacggaatcgaaagtcttctaatcccacgttcagttcaagaagatttcaacagaagaagaagtctccgatcaatctccgcggtattgccggaaggtgcgccggtagttgatcccagaacccacaggttaaaaaagccagttgcccctgttccggctggcgcacccccagttcttccaatctacgacgctttggctccaggtccatctctagctccagctccggctccgggaccaggtgggccgcgtggccacttcgacggaatggctcaagtcaaggactttatccaaaccctcgtgcattacggtgggtacaacgaaatggctgatattttagttaatctaacgtctctggccaccgagatgggtcggctcgtatcggaaggttatgtaattacggttttagccccgaacgacgaggccatggctaaactgactacggaccagcttagcgagcctggggcaccggaacagattgtgtactaccacatcataccagagtaccaaaccgaagagagtatgtacaatgcagttaggcgatttgggaaggttcgttacgataccttgaggctaccccataaggtcatggctcaagaagctgatggttcggtgaaattcggccatggcgatagccaggcttaccttttcgacccagatatctataccgatggccggatttcagttcagggcattgatggggttcttttcccacctaatgaggaccccaattcggagaagaaaacaactcctcttgttaaggttgtcaccaagcccaccagaagaggaaagttgatggaagtggcctgcaatgtgcttggtgtttttggtgtatcatcttcatgtcaataa

SEQ ID NO: 20 - DNA, 921 bp, Cannabis Sativa L.
atgaacaagcaagcaatcatttacttctcattcttgtttttgtgtttcttttaccactgcaaaggagccacaatattagctcactctccagcccaacctccatcaaaacatgtagctgctgcagccccaactaaagctaaggccttaaccccaacgaaagcgccaacagctttgccagtgcccgcggtggagccgccatctcaagtgccactcgtccaagcgccaccccacaaagccctttacacaccaacagacgtcaccaaaatcctcgaaaaagccggtatcttcagtgtcttcatccgcctccttaagagtacttctgtaagcattcagattgagaatcaactgaatgtatccaacacattgaccatttttgccccaacaaatggggcctttggtgctctgaaacctggcactctcaacacactctctaatgaagacaaagtccagcttgtccaataccatattcttccctctttggtttcactacaaaactttgagactcttagcaatcctgtacggacacaagctagtaataccaatgattttcctctaaatgttactgtggaaggaagctctgttaacatatctactggaattgttaatgctaccatttctggtactgtttatgaggataatcaacttgctatttacaaggttgataaggtgttgttgcctttgggaatcttcggtccgaaaccgaagacaaagcagcacctagcgccttctcccacaccattgaagccatctaaagatactaatgtgtcattaccttcttcctctactgaagaatccattagctcggatgtggatgaaggggacaagtcctcaaagtcgaaggctgctgtacttatgaataatggtgttgtaaacattggagttgttatgattgttgtaatcactatgtggggtcatttttag

SEQ ID NO: 21 - DNA, 951 bp, Cannabis Sativa L.
atggcaaaaaatcagattttgatcaccatttcgtgcctaattttattcctctccctcttccatactctgacattagctcagttgtcatcacctgcccaagcaccatcaacaacaccaaataagaagaatattcatccgtcatcacatcaatcgccagctcaatcgccggatcagccattggtggaagctccaccaacggctatttccaggagaaaaggacatcccaccaacattacagaaatcttggagaaagccggcgatttcagcctgtttattcgtctactaacaaacactgatgtcatcactcccatcgaaaatgatctcaactcttccaacaccgtcaccatttttgctccaaccaatggagcattctcagcccttaagactggaacactcaacactctcaccccccaacaaaaaacacaactagtccagtttcacatcatctcagccttcatacccctgcaaaacttccaaaccctaacaaaccctgttcatactcgagccattaacactcgcgacttcccattaaacattactagtactgacggatcttctgtcaacatatccactggtgttgtcaacacaaccatatctggaactgtttattccgataaccaactcgctatctatcgagttaacaatgtcttgcttcctatgaaaatttttgctcctaagaagttgatgagctcactggcaccgtctccagcacccgcggtggccttaaagcctaagaaaaaatcgatggcatctgctgaatcgtctgaatcaccctcgtcctcattaaaaccatcttcaacatctacatcatcatcgtcgccaacatcatcatcatcatcatccaatgatgatgaaacagtagccacttcgacaaatacatctggtgctgaacgggggccagtatttgccacgatctccattcttgttgctgttgttgcattgttcctatga

SEQ ID NO: 22 - DNA, 990 bp, Cannabis Sativa L.
  misc. feature 413: unknown
atggccaaatctctcttcttcttcttcattctttctatcttctcctcctctgctctcggctcttgcttgacccttttaaacgccgccgagattctctcaaactccggctacctctcaatgtccttgacccttaaaatcgcctctcaaaccattaaacacgattcaccgaccgccaccgtctttgctccggcagaccaagctttcgtcaagtcaggccaaccttccctgtttctccttcgccgccacgtgtcccccgtaaaactctcactcgaaacactcaagactcttcctcgtggctcagtaatacccaccatggtccccgaccaccctctaatcgtcaccgcttctctctccggcgacggctacatctccataaacgacgtcaggatcaacgagaaagccgtttttggtgntgggttcgtagctctgtacggtatagacgagttcattgactcatctttcttccggactgatcagccgccgtccccagctccggctccggcgcctgccccgttccacggcaagacagagtcttttgcttcggttgctgagtttctcaggtcgagaggttactatataatggccacgcttcttgatgcccaattgaccgggctcggtgacgggaccaaattgaccatctttgctccggtggaccgaactttcgattactatgcatcgaatataagcgattacgctttgattttccggagacatgttgtgccgagattgatgacgtggcaggacttgattggaagccaagttgttgggacaaagcttccgactttttcaaggggttttatgatcgaggtgagggtgtcaagtgatggtattcctatgcttaacgatgcaccaattgtgtttcaagatatatatcgaagtcaaaggttgattgttcatggccttaatgggtttctcaagcctttcactgatcaagaatggaatcaagattctttctctaatggatttgttggtgatgattctcatggataa

SEQ ID NO: 23 - DNA, 1068 bp, Cannabis Sativa L.
atggctcattgttgcaccggctcgtggcgtgcaccggtctacttcaccgtgtctgtaatcttggccttcatagccatctcaacatcaatgcactccaaaaccgaacatccatcctccccaaccaaactcaatttccatgaactctccctcaacgcctccaaagccttaagaaaagctggcttcaacgtcatggccacccttcttcaagtctcccctgagatcttcctcttatctccaaactcaactatctttgccattcaagatgatgccatctccaactcttcactcccaccatggcttttgagaaacctccttcaatatcacacctctcctctcaaccttcccatgaaagacctcttaaagaagcctcgagggagttgcttgccgacccttcaccgccaaaagaacattgccatcactaacatcgacttcaaagaaacaacagttgatatcaacaacgtttcagtgactcatcctgatgtatttcttgcagaaaccatttccgtccacggtgttcttgagcccttttcttcgttggatcctgaagatgttcatcagggttggaactctatccaagcccccacttgcaacgcaatgtctgttctagtttcggatgctgtgaaatccaccaacatggttgaatggtcgtggattgtcaggttgctgagctcgaacggattcgttcctttcgctataggattaaattctgttcttgaagagattcttaaagactacaagggattgaattctgtaacaatctttgctcctccaaatttgcagtctttgacatctccttcacccctactcaaaagaacagtatggtttcacatacttcctcagaggttaacgtataaagaacttactgcattgcctgctggaaccttgctcatgacattggttcgtgatctatctctcgaggttgcagggacagcaggtttcaaaggtggactaatcataaatggaatcgagatcgtggcgcccgacattttcacttcaaagaagttcaccgtgcatgggatttctcgagcttttgaggttgctgatcaggtagctgcaatcggtacatag

SEQ ID NO: 24 - DNA, 972 bp, Cannabis Sativa L.
atggcggcttcacttctcatctctctaactcttctctcatttctttctctctcctcctcactcccctcaaacaccattatcgatgcctccgagattctctccgactctggcttcgcttccatggctcttactctggatctcgtctcccaaaccctaacccaaaggactccttctctcacgatatttgctccggccgatgacgcctttaagagatcagggcaacccgctctatctctcctccgttaccacttctgccccctcactttgccgctggagaccctcaaatcgcttccctctgggaccaagatcccgactctgttacccggccgcaccttgaccgtcactcattcctcctccacctctgaaatttcactgaacaatgtcaaaatcagtagaagatttccgatcttcgatgatggttctctgattgtcttcggagttccagagttcttcgatcccaattttcaagctcccggacccggtaatagcccgaggtttggtccaagatgcaaatcgttgccatcgaaggctgccgccatggggtttccgggagcttcttggtttaaagaagccagtagagatttgaggtccaatgggtactcatccatggcttcgtttctggatttgcagttgttggggttcaacaaggacccgactacaatgactgtgtttgctcctaatgacatggccatggcgaatcgtccaaccgatcaggctcaagacccatcaatctttctacgacatgtcgttccttgcaagcttctttggagtgatttgatcaatttcactgatggaacggtgttgcctacatactcggatgggttcaccatcaccattaccagatcgggtagtacattgatgattaatggaattcctgtaaccgtttccaacctccattatagtgattctgtcgttgttcatggcctaaatgagcttcttactggacaagcaactacatctgggtcaggttaa

SEQ ID NO: 25 - DNA, 606 bp, Cannabis Sativa L.
atgaagatgaacagaggtcggtttttgaagacttcgatctccttcgtatggttggtggtgctttttggctgcctttttgtggttctaatttctgttcttaggcttccagaggtgtcaaatagtagaaaggcaattgggttgtatcataatacaaaaaccagagaaagttcagagtctagctctattgggaaatttggagagaagatgattgaaatgttgcctgaagatcttgctttcactgtttttgttccttcagagagagcctttgaacgagatttaaggctaaggaaaaacgaaagtctggttgagaagatgaatgatgatacttatgctgtaatttctagagttctgggattctcggctgttccaaggacaattatcacagatgatgtatcttctggtgaggagattttgtatgactctatatctgggtttgtattgtatatttccaaagatgtggatggaatgttagtggttaacagagttcgttctgaaaaagtagatatcaaaaggaacaaaattgttgtacatgtaatggatggggttatcatggatgctgaatttgaagaatcagttcagcctgatgatgaggatgaagaaaagtga